(19) United States
(12) Patent Application Publication
(10) Pub. No.: US 2022/0208390 A1
(43) Pub. Date: Jun. 30, 2022

(54) VACCINATION RECORD

(71) Applicant: Change Healthcare Holdings, LLC, Nashville, TN (US)

(72) Inventors: Gautam M. Shah, San Francisco, CA (US); Anthony David Guido, Corona, CA (US); Paul Olstad, Elk River, MN (US); John Foss, Colorado Springs, CO (US); Brad Crosslin, Keller, TX (US); Shanker Rakasi, Maple Grove, MN (US); Brendan Pittman, Austin, TX (US); Sergey Polyakov, Norwalk, CT (US)

(21) Appl. No.: 17/519,067

(22) Filed: Nov. 4, 2021

Related U.S. Application Data
(60) Provisional application No. 63/132,848, filed on Dec. 31, 2020.

(51) Int. Cl. G16H 50/70; G16H 50/20; G16H 10/00

(52) U.S. Cl. CPC G16H 50/70; G16H 10/00; G16H 50/20

(57) ABSTRACT

A vaccination record is provided. Initially, when a patient gets a vaccine, such as a COVID-19 vaccine, an indication of the vaccine is stored in an electronic file or data structure associated with the patient called a vaccination record. The record may include information such as the type of vaccine that was administered, the date that the vaccine was administered, and an indication of the entity (e.g., clinician or healthcare practitioner that performed the vaccine). The record of the vaccine may be created in response to a billing submission by the entity. Later, the patient may use the vaccination record to provide proof to third-party verifiers that the patient in fact received the vaccine.

### CROSS-REFERENCE TO RELATED APPLICATIONS

This application claims priority to U.S. Provisional Patent Application Ser. No. 63/132,848, filed on Dec. 31, 2020, and entitled “Vaccine Passport”. The disclosure of which is incorporated by reference in its entirety.

### BACKGROUND

Many providers of activities and services such as airlines, venues, gyms, restaurants, and salons would like to require that their customers be vaccinated for diseases such as COVID-19. However, there is currently no way to maintain an electronic record of immunizations and test results in a way that is searchable, interoperable, verifiable, and accurate.

### SUMMARY

In one embodiment, a vaccination record is provided. Initially, when a patient gets a vaccine, such as a COVID-19 vaccine, an indication of the vaccine is stored in an electronic file or data structure associated with the patient called a vaccination record. The record may include information such as the type of vaccine that was administered, the date that the vaccine was administered, and an indication of the entity (e.g., clinician or healthcare practitioner that performed the vaccine). The record of the vaccine may be created in response to a billing submission by the entity. Later, the patient may use the vaccination record to provide proof to third-party verifiers (e.g., schools, airlines, restaurants, venues, job locations, and hotels) that the patient in fact received the vaccine. In one example, the patient may be provided with a QR code or other link that may be shown or electronically submitted to the third-party verifier. The third-party verifier may then use the QR code or link to retrieve proof that the patient was vaccinated along with optional challenge information for the patient. The third-party verifier may use the challenge information to verify that the patient is who he or she purports to be.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIG. 1 is an illustration of an exemplary environment for creating and providing a vaccination record;

FIG. 2 is an illustration of a method for creating a vaccination record in response to determining that a patient received a vaccine;

FIG. 3 is an illustration of a method for proving a vaccination record to a patient;

FIG. 4 is an illustration of a method for verifying a vaccination record;

FIGS. 5-11 are illustrations of example user interfaces for using vaccination records; and

FIG. 12 shows an exemplary computing environment in which example embodiments and aspects may be implemented.

### DETAILED DESCRIPTION

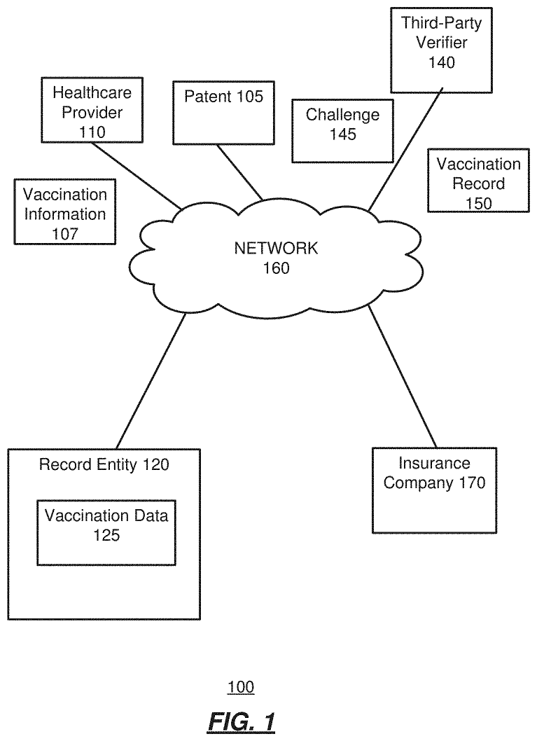

FIG. 1 is an illustration of an environment 100 for providing a vaccination record 150. As shown, the environment 100 includes one or more patients 105, one or more healthcare providers 110, one or more record entities 120, one or more third-party verifiers 140, and one or more insurance companies 170 in communication through a network 160. The network 160 may be a combination of public and private networks and may use a variety of wired and wireless networking technologies. An example network 160 is the Internet.

The record entity 120 may be an entity that maintains vaccination data 125 for one or more patients 105 (also referred to as users). Depending on the embodiment, the record entity 120 may maintain vaccination data 125 for each patient 105 along with information about the patient such as date of birth, telephone number, name, and address. Other information may be included.

The vaccination data 125 maintained for each patient 105 may include information about each vaccine that the patient 105 has received. The vaccination data 125 for a vaccine may include the name of the vaccine, the manufacturer of the vaccine, a batch or lot number associated with the vaccine, the name of the clinician and/or healthcare provider 110 that administered the vaccine, the date when the vaccine was administered, and whether the vaccine is a multi-part vaccine or requires one or more boosters. Other information may be included.

The record entity 120 may use the vaccination data 125 for a patient 105 to create what is referred to herein as a vaccination record 150. The vaccination record 150 may be an electronic record or file that a patient 105 may provide as proof to one or more third-party verifiers 140 that the patient 105 had a particular vaccine. The third-party verifier 140 who receives a vaccination record 150 (either from patient 105 or the record entity 120) may then use the vaccination record 150 to verify that the patient 105 received the desired vaccine.

Examples of third-party verifiers 140 may include, but are not limited to, airlines, hotels, venues, schools, ticket providers, or any other entity who may wish to limit their facilities or services to patients 105 who have received a particular vaccine. For example, an airline may desire to exclude passengers who have not yet received a COVID-19 vaccine. Accordingly, the airline may require that each passenger provide a vaccination record 150 that can be verified by the airline before boarding the airplane.

In some embodiments, the vaccination record 150 may be downloaded onto a computing device associated with the patient 105. Example computing devices include mobile computing devices (e.g., smart phones), table computing devices, video game consoles, and laptop or desktop computing devices.

The patient 105 may request their vaccination record 150 from the record entity 120. For example, the patient 105 may send a text to the record entity 120 that indicates that they would like to receive the vaccination record 150 at their mobile computing device (or other computing device). In response, the record entity 120 may generate a vaccination record 150 for the patient 105 and may provide the vaccination record 150 to the patient 105. The patient 105 may then store the vaccination record 150 on their mobile device, may print the vaccination record 150, or may share the vaccination record 150 with one or more third-party verifiers 140.

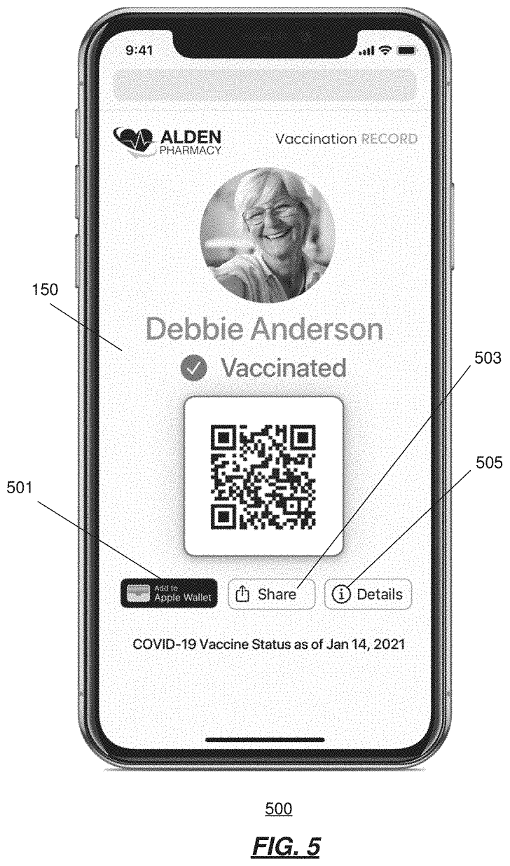

In some embodiments, the vaccination record 150 may be a code (e.g., QR code) that may encode a link or pointer to some or all of the vaccination data 125 (or representations thereof) associated with a patient 105. For example, FIG. 5 includes an illustration of such a vaccination record 150.

Each vaccination record 150 may include a QR code that encodes a link to some or all of the vaccination data 125 associated with the patient 105. Each record 150 may be implemented as a webpage that is accessed by the patient 105 using a browser associated with their mobile device. Alternatively, the record 150 may be part of a stand-alone application or “app” that is downloaded by the patient 105 and created by the record entity 120.

Returning to FIG. 1, the patient 105 may present the vaccination record 150 when requested by a third-party verifier 140. Continuing the example above, an airline may request proof that the patient 105 has a particular vaccine. The patient 105 may cause the vaccination record 150, including the QR code, to be displayed on their mobile device. The airline employee (i.e., third-party verifier 140) may then scan the QR code using their own mobile device and may be redirected to a webpage provided by the record entity 120. The webpage may identify the patient 105 along with one or more vaccines that were recorded for the patient 105. The airline employee may then verify that the patient 105 received a particular vaccine via the webpage.

In some embodiments, the vaccination record 150 may further provide a challenge 145 that may be used by the third-party verifiers 140 to verify that the patient 105 that presents the vaccination record 150 is in fact the patient 105 associated with the record 150. One example of a challenge 145 is the birthdate of the patient 105 or other information that is known about the patient 105 by the record entity 120. Other known information that could be used as challenge 145 include a phone number associated with the patient 105, an address associated with the patient 105, and a date when the patient 105 last received a vaccine. Other example challenges 145 include codes or answers to secret questions provided by the patient 105 (“What was the name of your high school?”). The patient 105 may have provided the answers to the challenge questions when setting up the vaccination record 150 with the record entity 120.

If the patient 105 answers the challenge 145 correctly, the third-party verifier 140 may then determine that the patient 105 is vaccinated. The patient 105 may then be allowed to participate in the particular activity or service provided by the third-party verifier 140 (e.g., get on the plane, attend the concert, enter the restaurant, etc.).

The patient 105 may share their vaccination record 150 in a variety of ways such as printing, email, text, or through a social networking application. The patient 105 may also share their vaccination record programmatically with another application using an API to securely share data, or by generating data in a specific format, e.g., a C-CDA for sharing with a specified computing system.

In one example, the patient 105 may share their vaccination record 150 with third-party verifiers 140 electronically though the network 160. For example, a patient 105 may be purchasing a ticket to a concert from a third-party verifier 140 through the internet. Before finalizing the ticket purchase, the third-party verifier 140 may ask the patient 105 to provide their vaccination record 150 so that the verifier 140 can verify that the patient 105 had a particular vaccine (e.g., COVID-19 vaccine). In response, the patient 105 may enter a code associated with their vaccination record 150, or the QR code associated with the vaccination record 150. The third-party verifier 140 may then use the code to retrieve the vaccination data 125 from the record entity 120. Once verified, the patient 105 may complete the purchase of the tickets.

Alternatively or additionally, in response the patient 105 may go to a website associated with the record entity 120 and may provide specific information to grant consent to share their vaccination record 150 with the third-party verifier 140. After providing the consent, the record entity 120 may provide the vaccination record 150 (or QR code equivalent) to the third-party verifier 140 via the network 160.

In some embodiments, the vaccination record 150 may be implemented as a standalone app that is downloaded to the mobile device (or other computing device) associated with the patient 105. The patient 105 may then use the app to control what vaccines are listed in the vaccination record 150 and to share the vaccination record 150 with one or more third-party verifiers 140. When such a standalone app is used, the app may verify the patient 105 using the challenge 145, rather than the third-party verifier 140.

The record entity 120 may build and maintain the vaccination data 125 that is used to implement the vaccination record 150 using information received from a variety of sources. The record entity 120 may receive vaccine information 107 from a healthcare provider 110 when a patient 105 receives a vaccine. The vaccination information 107 may include information about the vaccine such as the type of vaccine, when and where the vaccine was administered, who administered the vaccine, and may identify the patient 105.

In some embodiments, the record entity 120 may be located at, or may be part of, a healthcare clearinghouse that receives and processes claims from a healthcare provider for one or more insurance companies 170. When a claim for a patient 105 that is received from a healthcare provider 110 indicates that a vaccine was administered, the healthcare clearinghouse may pass the claim to the record entity 120. The record entity 120 may then extract the vaccination information 107 from the claim and may add the vaccination information 107 to the vaccination data 125 for the patient 105. Healthcare clearinghouses may further include state health registries and EHRs, for example

In some embodiments, where the vaccine is a multi-part vaccine like the COVID-19 vaccine, the record entity 120 may periodically remind the patient 105 regarding when the patient 105 is due to receive a next dose of the vaccine, or a booster dose of the vaccine. For example, after the patient 105 receives the first dose of the vaccine the record entity 120 may text, or otherwise send electronic messages to the patient 105, reminding the patient 105 of an upcoming date to receive the next dose. In another example, the vaccination record 150, when viewed by the patient 105, may display the date when the second dose is due, or may display a “countdown timer” that counts down to when the second dose is due. The timer may be displayed to the patient 105 in a webpage or an application running on their mobile device. The countdown timer may also be used for vaccines that need to be updated regularly, or for booster doses of the vaccine. Other methods for reminding the patient 105 may be used.

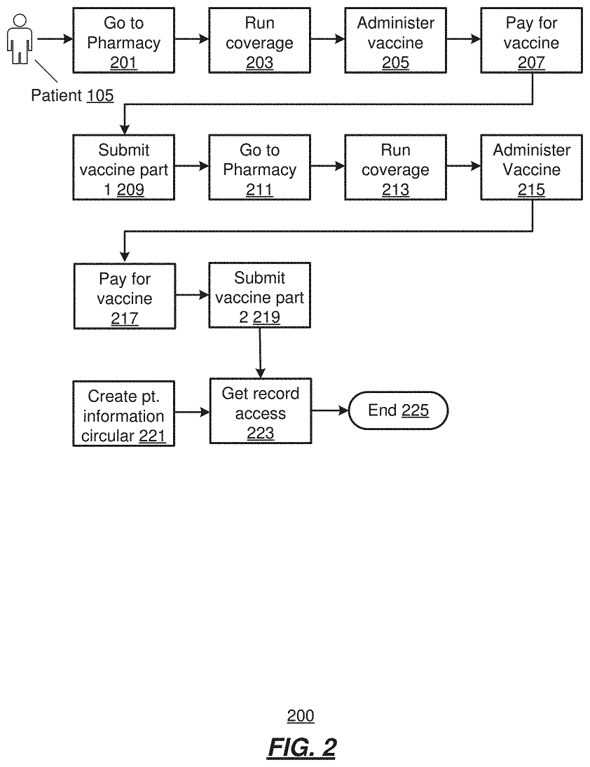

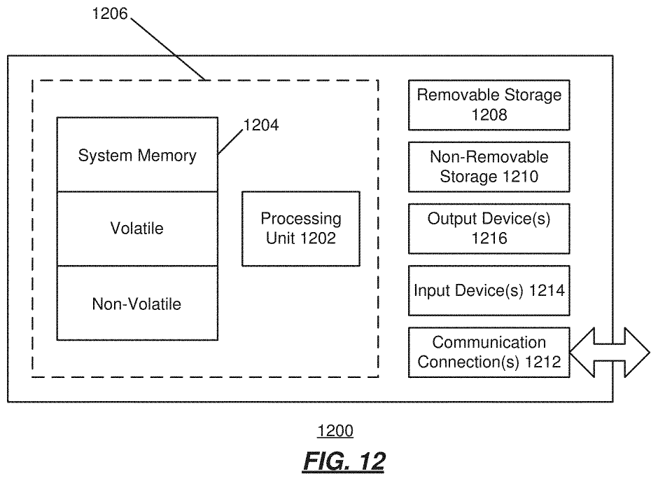

FIG. 2 is an illustration of an exemplary method 200 for receiving a vaccine and for adding the vaccine to the vaccination record 150. The method 200 may be implemented by one or more computing devices such as the computing device 1200 illustrated with respect to FIG. 12. Note that that some of the steps of the method 200 may be performed in a different order than shown and described.

At 201, the patient 105 goes to a healthcare provider 110 to receive a vaccine. In the example shown, the healthcare provider 110 is a pharmacy and the vaccine is a two-part COVID-19 vaccine. Other vaccines and healthcare providers 110 may be supported.

At 203, the pharmacy runs coverage for the vaccine. Running coverage may include determining the insurance company 170 or provider associated with the patient 105 and determining that the insurance company 170 will cover the vaccine.

At 205, the the vaccine is administered. The vaccine may be administered by a clinician at the pharmacy or the healthcare provider 110. Where the vaccine is a COVID-19 vaccine, the first part or dose of the vaccine is administered.

At 207, the patient pays for the vaccine. The payment may be made by the patient 105 (i.e., cash or credit card payment) or the payment may be made indirectly by the insurance company 170 or government agency. Depending on the vaccine and insurance company 170, the patient 105 may provide a co-pay or partial payment for the vaccine.

At 209, that the first part of the vaccine was administered is submitted. A record of the administration of the first part or dose of the vaccine may be submitted to an entity 120 associated with the vaccination record 150. In some embodiments, the record entity 120 may maintain vaccination data 125 of each vaccine received by the patient 105 or user. The vaccination data 125 may include information such as the type of vaccine that was administrated, the date that the vaccine was administered, the healthcare provider or pharmacy that delivered the vaccine, and the dose of the vaccine that was administered. Other information may be included in the vaccination data 125.

In some embodiments, the submission of the vaccine may be recorded in response to various triggers or actions that take place during the processing of the insurance claim or coverage related to the vaccine by the pharmacy or healthcare provider 110. For example, when the pharmacy submits the claim related to the vaccine to a claims administrator for the patient 105, vaccination data 125 that indicates the vaccine may be automatically generated and associated with the patient 105. Alternatively, the vaccination data 125 may be created in response to information about the vaccine and patient 105 provided directly by the pharmacy or healthcare provider, or the vaccination data 125 may be created in response to information about the vaccine provided directly by the patient 105. Depending on the embodiment, after the vaccination data 125 is created an electronic message may be sent to the patient 105 indicating the availability of the vaccination record 150. As another example, the vaccination data 125 may be generated from claim payment data (i.e., data showing that a claim was paid).

After some time has passed, at 211, the patient 105 may return to the pharmacy or healthcare provider 110 for the second part or dose of the vaccine. The second part of the vaccine may be administered, and payment may be processed similarly as described for the first dose (e.g., steps 213; 215; and 217). In addition, vaccination data 125 corresponding to the second part or dose of the vaccine may be created in response to information received from the pharmacy or claims administrator (e.g., step 219).

At 223, after the patient 105 completes the second dose of the vaccine (or other dose depending on the type of vaccine), the patient 105 may be provided access to their vaccination record 150. For example, the patient 105 may be provided with a short code and a phone number that the patient 105 can use to request access to their vaccination record 150. As another example, the patient 105 may be emailed a link that they can use to access the record 150 or may be provided with a link to download an app through which the patient 105 can access the record 150. Depending on the embodiment, the healthcare provider 110 may provide an information circular or brochure that explains the vaccination record 150 and how to access or use the vaccination record 150 (e.g., the step 221). The method 200 may then exit at 225.

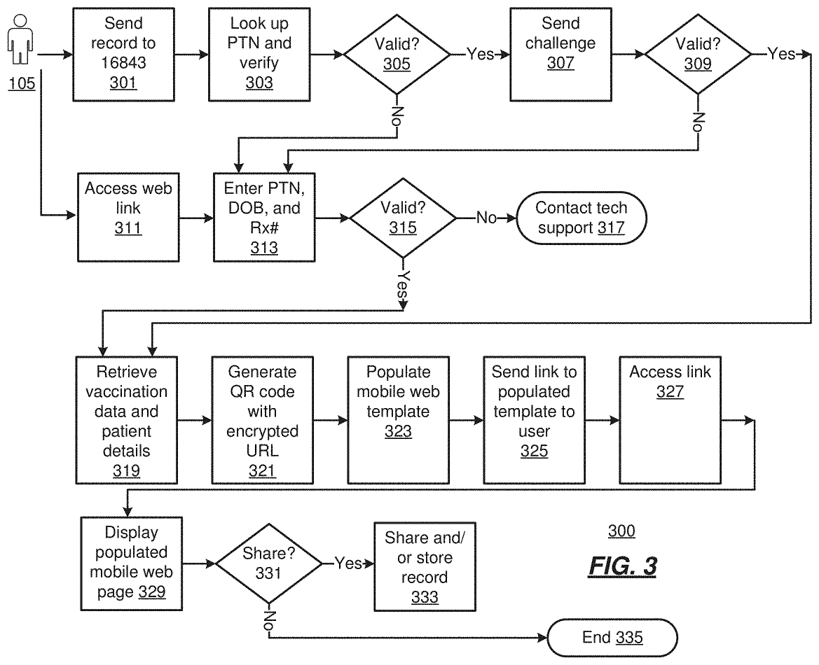

FIG. 3 is an illustration of an exemplary method 300 for providing a vaccination record 150 to a patient 105. The method 300 may be implemented in part by a mobile device associated with a patient 105 and one or more computing devices such as the computing device 1200 illustrated with respect to FIG. 12. Note that that some of the steps of the method 300 may be performed in a different order than as shown and described.

A patient 105 using a computing device such as a laptop, desktop, smartphone, tablet, or non-smart phone (e.g., a flip phone) may desire to receive a copy of their vaccination record 150 so that they can prove to a third-party verifier 140 that they received one or more required vaccines. For example, the patient 105 may be required to show proof of vaccination prior to boarding an airplane or may be required to provide proof of vaccination prior to purchasing a concert ticket on the internet.

With reference to FIG. 3, at 301, the patient 105 may request their vaccination record 150 by texting a codeword to a specified number. The codeword and number may have been provided to the patient 105 when they received their vaccine. In the example shown in FIG. 3, the patient 105 may text the codeword “record” to the number “16843”. Other codewords and numbers may be used.

At 303, the record entity 120 responsible for maintaining the vaccination records 150 receives the request and determines if there is a patient 105 or vaccination record 150 associated with the phone number. If there is not a valid patient 105 associated with the request (305), the method 300 continues at 313. Else, the method continues at 307.

At 307, a challenge 145 is provided back to the patient 105. The challenge 145 may be provided via text, email, phone call or any other method. The challenge 145 may ask the patient 105 to provide “secret” information that was previously provided by the patient 105 when creating the vaccination record 150 or an associated user account. Alternatively or additionally, the challenge 145 may be the birthdate of the patient 105. Any method for challenging or authenticating a patient 105 may be used.

At 309, whether the challenge is valid is determined (i.e., did the patient 105 provide the correct information). If the challenge is valid, the method 300 may continue at 319. Else, the method 300 may continue at 313.

In some embodiments, the patient 105 may request the vaccination record 150 using a link or other address provided on a webpage. For example, when purchasing a ticket to a concert or airline online, the patient 105 may be presented with a URL that they can use to request a vaccination record 150. Such an embodiment is shown in FIG. 3 beginning at step 311.

At 313, the patient 105 is asked to provide information that may be used to identify the patient 105. The additional information may include date of birth, address, phone number, account number, and/or social security number. Other information may be requested.

At 315, whether the additional information is valid is determined (i.e., did the provided information match a known patient 105 or a user profile). If not, the patient 105 may be directed to contact online technical support at 317. Else, the method 300 may continue at 319.

At 319, vaccination data and other patient information is retrieved. The information may be stored by the record entity 120 as part of the vaccination data 125. The retrieved vaccination data 125 may include information about each vaccine that the patient 105 received such as the name of the vaccine, the date the vaccine was received, the number of doses that the patient received, and the clinician or provider that administered the vaccine. Depending on the embodiment, the retrieved vaccination information may only include information about a particular vaccine associated with the patient 105. For example, the patient 105 may have specified or identified the vaccines that they would like to include in their vaccination record 150 either when they requested the vaccination record 150 or through one or more settings associated with a user account or a profile associated with the patient 105.

At 321, a QR code with an encrypted URL is generated. The QR code may include an encrypted link or URL that points to some or all of the vaccination data downloaded for the patient 105. Any method for generating QR codes may be used. Other types of codes may also be used such as bar codes, for example.

At 323, a web template is populated with the generated QR code. The web template may be optimized for viewing on mobile devices, or more generally applicable web templates may be used.

At 325, a link to the populated web template is provided. The link may be provided to the patient 105 that requested the vaccination record 150. Depending on the embodiment, the link may be provided to the patient 105 at the same device that the patient 105 used to request the vaccination record 150.

At 327, the patient 105 may access the vaccination record using the link. Depending on the embodiment, the patient 105 must first check or select a box that indicates that the patient 105 agrees to terms and conditions associated with use of the vaccination record 150.

At 329, the populated webpage is displayed to the user. The populated webpage may be displayed to the patient 105 on the device that they used to request the vaccination record 150. The populated webpage may further include one or more links or user interface elements that allow the patient 105 to store or share their vaccination record 150.

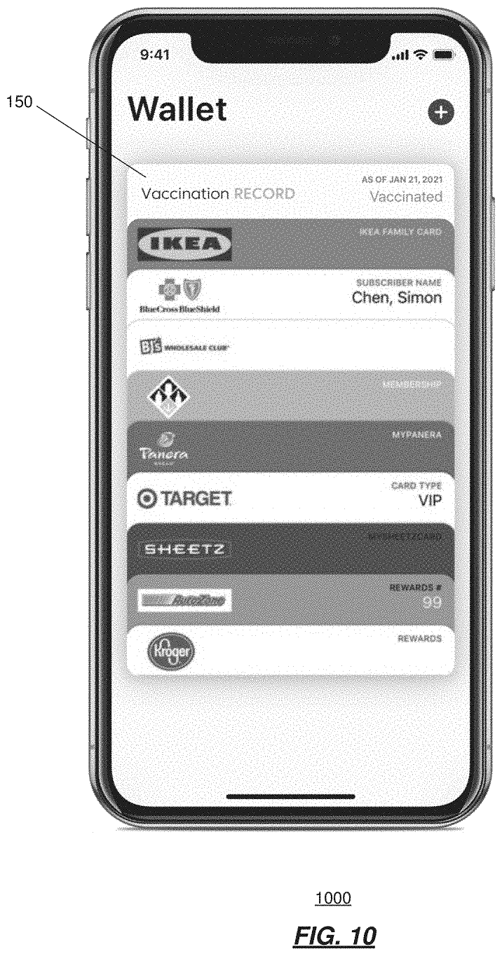

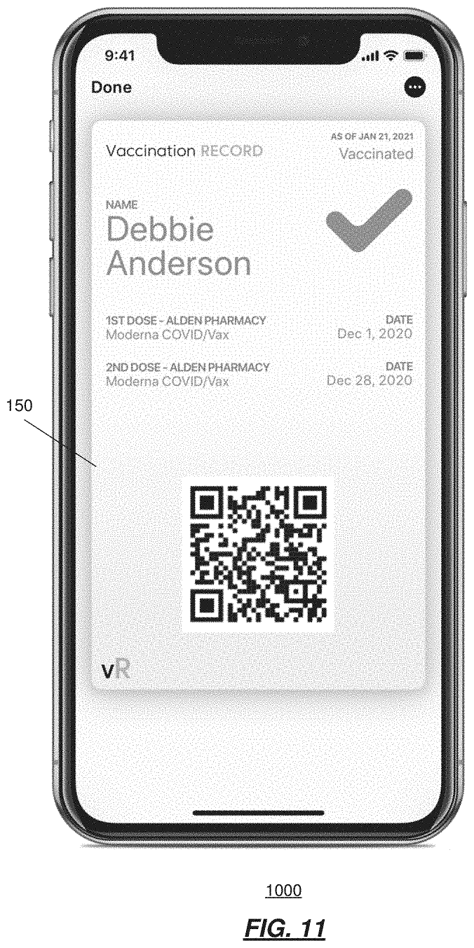

In some embodiments, the populated webpage may include a button that allows the patient 105 to store their vaccination record 150 in a mobile wallet. Examples include Apple Wallet or Google Pay. After storing the vaccination record 150, the patient 105 can display the vaccination record 150 (including QR code) on their mobile device (or other computing device) using their mobile wallet. Examples of a mobile wallet including a vaccination record 150 are illustrated in FIGS. 10 and 11.

In some embodiments, the populated webpage may include a button that allows the patient 105 to share their vaccination record 150 using a variety of electronic messaging technologies. Examples include SMS and email.

In some embodiments, the populated webpage may include a button that allows the patient 105 to print their vaccination record 150 (including QR code). The patient 105 may select among a variety of local and remote printers to print their vaccination record 150.

In some embodiments, the populated webpage may include a button that allows the patient 105 to share their vaccination record 150 with one or more pre-selected partners or third-party verifiers 140. For example, the populated webpage may include buttons to share the vaccination record 150 with partners such as ticket providers, airlines, venues, or any other location or entity who may desire to limit their services to users who have had a particular vaccine. Each partner may have been vetted and may have agreed to certain conduct or procedures related to the handling of the vaccination records 150.

In some embodiments, the populated webpage may include a button that allows the patient 105 to share their vaccination record using a beacon. For example, the mobile device associated with the patient 105 may use Bluetooth, or another wireless technology, to generate a beacon signal that indicates that the vaccination record 150 is available. Other users or entities may then respond to the beacon signal to receive the vaccination record 150. Alternatively, or additionally, the beacon may broadcast a signal that indicates the vaccination status of the patient 105 (e.g., vaccinated or not-vaccinated) with respect to a particular vaccine.

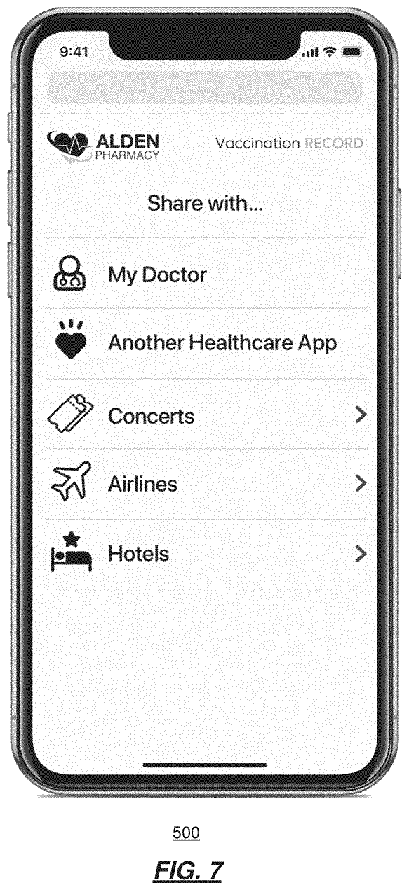

At 331, the patient 105 may determine whether to share or store their vaccination record 150. If the patient 105 determines to share or store the vaccination record 150, the method 300 may share or store the vaccination record at 333. Otherwise, the method 300 may then exit at 337. An example of vaccination record 150 sharing is shown in FIG. 7.

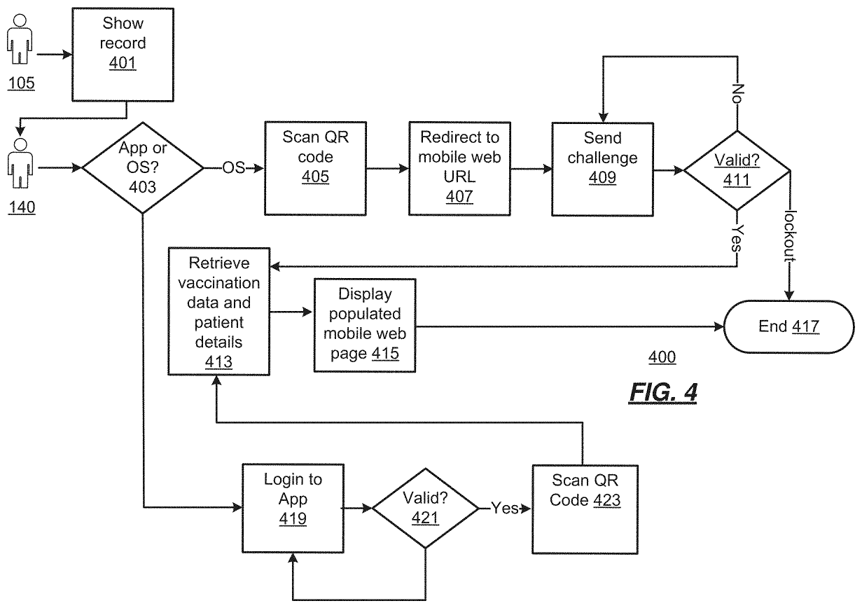

FIG. 4 is an illustration of an exemplary process for verifying a vaccination record received from a patient 105 by a third-party verifier 140. The method 400 may be implemented in part by a mobile device or a computing device such as the computing device 1200 illustrated with respect to FIG. 12. Note that that some of the steps of the method 400 may be performed in a different order than as shown and described.

At 401, a patient 105 may show their vaccination record 150 to a verifier 140. The verifier 140 may be a third-party verifier 140 (e.g., airline, school, restraint, venue, or amusement park) who is interested in verifying that the patient 105 has had a particular vaccine.

The patient 105 may show or provide their vaccination record 150 to the verifier 140 in a variety of ways. In one example, the patient 105 and the verifier 140 are together in the same location and the patient 105 may show their vaccination record 150 by displaying a QR code on their mobile device, by providing a piece of paper on which the QR is printed, or by activating a beacon on their mobile device that causes the vaccination record 150 to be provided to nearby devices.

In another example, the verifier 140 and the patient 105 are in different locations, and the patient 105 may show their vaccination record 150 by causing the QR code, or other unique identifier, to be electronically provided to the verifier 140. For example, the QR code may be provided to the verifier 140 via email. Alternatively, the patient 105 may speak a code or identifier of the vaccination record 150 to the verifier 140 via the telephone or may enter the code into a field in a webpage provided by the verifier 140.

At 403, how the method proceeds may depend on whether the verifier 140 is scanning the QR code using their mobile device “OS” or whether the verifier 140 is using another app to verify the record 150. If the verifier 140 is using their mobile device the method 400 may continue at 405. Else the method 400 may continue at 419.

At 405, the verifier 140 scans the QR code associated with the vaccination record 150. For example, the verifier 140 may open a camera app of their mobile device and may maneuver their device so that the QR code of the vaccination record 150 is visible on the screen of their mobile device.

At 407, the mobile device of the verifier 140 is redirected to a URL. The mobile device may automatically open a web browser at the URL encoded into the QR code.

At 409, a challenge 145 is provided for the verifier 140 to challenge the patient 105 with. Depending on the embodiment, the challenge 145 may be a question that the verifier 140 is provided to ask the patient 105 that is presenting the vaccination record 105. The question may be for the birthdate of the patient 105, a telephone number of the patient 105, or some other question whose answer may only be known by the patient 105 such as “Where/When did you receive the vaccine?” or “What was the name of your first pet?”. Other questions may be used.

At 411, the verifier 140 may enter the answer provided by the patient 105, and the answer may be verified. If the answer was correct and successfully verified, the method 400 may continue at 413. Else, the method 400 may return to 409 and a new challenge may be sent to the verifier 140. If the number of failures exceeds a threshold, the patient 105 may be locked out from providing the vaccination record 150 until some further action is taken by an administrator (e.g., account reset) and the method may exit at 417.

At 413, vaccination data 125 and other patient data is retrieved. The vaccination data 125 may include information about one or more vaccines such as the type of vaccine, the date when the vaccine was administered, the location of where the vaccine was administered, the clinician that administered the vaccine, and how many doses of the vaccine were administered. Other information may be included.

At 415, the vaccine and other patient data is displayed to the verifier 140 as the vaccination record 150. The verifier 140 may then view the data and may determine that the patient 105 was in fact vaccinated. After the patient is verified, the method 400 may exit at 417.

Where the verifier was using an application to verify the patient 105, the verifier 140 may login to the application at 419 by providing credentials such as a username and password. If the credentials are verified as valid (421), then the method may continue at 423. Else, the method 400 may return to 419 and/or exit at 417.

At 423, the verifier 140 scans the QR code associated with the vaccination record 150 using the application. Alternatively, the verifier 140 may manually enter a code or other identifier into the application. The number may be extracted from the QR code or displayed along with the QR code by the patient 105. After the QR code is scanned, the method 400 may continue at 413 and may proceed as described above.

FIG. 5 is an illustration of an example user interface 500 including a vaccination record 150. As shown, the user interface 500 is displayed by a mobile computing device. The displayed vaccination record 150 indicates that the patient 105 has been vaccinated for COVID-19 and includes information about the vaccine that the patient 105 received. The displayed vaccination record 150 includes a wallet button 501, a share button 503, and a details button 505. The record 150 further includes a QR code that can be scanned by a third-party verifier 140.

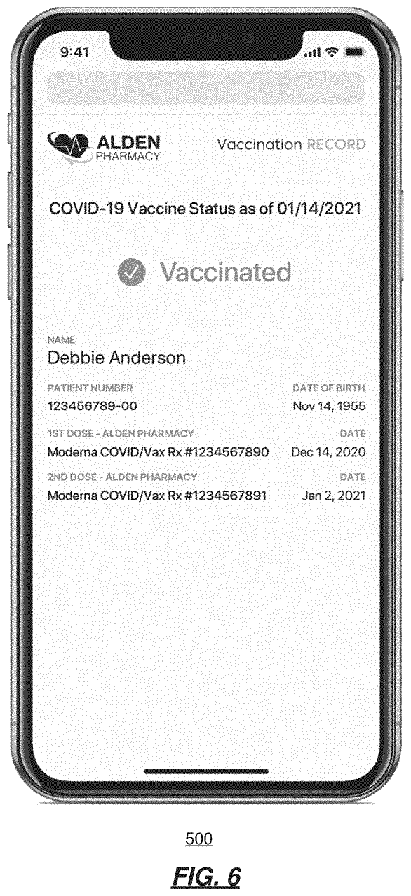

FIG. 6 is an illustration of the user interface 500 after the patient 105 has pressed the details button 505 from FIG. 5. As shown, additional details about the vaccination record 150 are displayed such as the number of doses received, where the patient 105 received each dose, the lot number associated with each dose, and the date when each dose was administered.

FIG. 7 is an illustration of the user interface 500 after the patient 105 has pressed the share button 503 from FIG. 5. As shown, various entities with which the patient 105 may share the vaccination record 150 with are displayed.

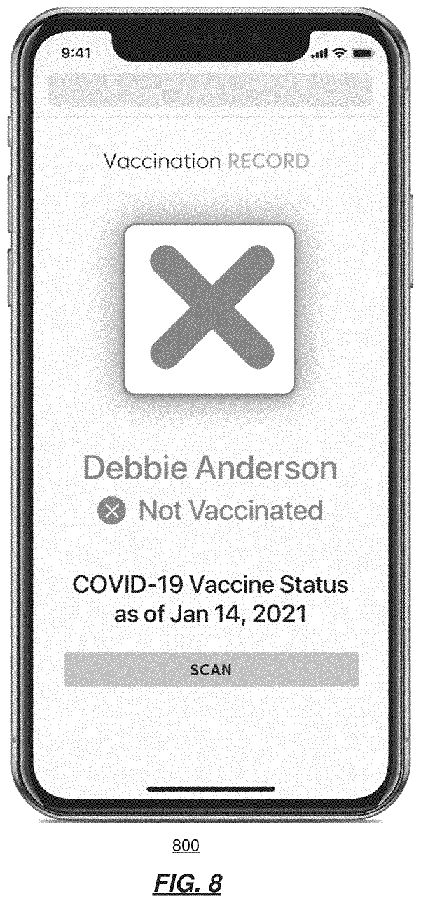

FIG. 8 is an illustration of an example user interface 800 as seen from the point of view of a third-party verifier 140 when a patient 105 has not been vaccinated or vaccination status cannot be determined. The user interface 800 is displayed on a mobile computing device associated with the third-party verifier 140. The third-party verifier 140 may have scanned the QR code of the vaccination record 150 displayed by a patient 105. In response, on the user interface 800 is shown an indication that the patient 105 has not been vaccinated as of the date indicated. Note that the indication does not include any personal information about the patient 105 besides name, vaccine status, and the current date.

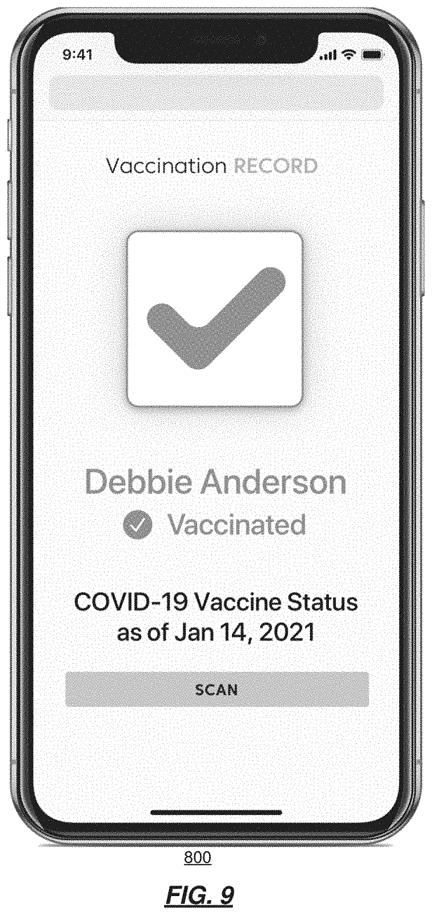

FIG. 9 is an illustration of the user interface 800 as seen from the point of view of the third-party verifier 140 when the patient 105 has been vaccinated. The third-party verifier 140 may have scanned the QR code of the vaccination record 150 displayed by a patient 105. In response, on the user interface 800 is shown an indication that the patient 105 has been vaccinated as of the date indicated but does not include any personal information about the patient 150 besides name, vaccine status, and the current date.

FIG. 10 is an illustration of an example user interface 1000 displaying a vaccination record 150 in a digital wallet 1005. In the example shown, the record 150 is one of many cards that are displayed as part of the digital wallet 1005 of the patient 105. FIG. 11 is an illustration of the example user interface 1000 after the patient 105 selected the vaccination record 150. As shown, the wallet 1005 of the user interface 1000 has been replaced with the selected vaccination record 150.

FIG. 12 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 12, an exemplary system for implementing aspects described herein includes a computing device, such as computing device 1200. In its most basic configuration, computing device 1200 typically includes at least one processing unit 1202 and memory 1204. Depending on the exact configuration and type of computing device, memory 1204 may be volatile (such as random-access memory (RAM)), non-volatile (such as read-only memory (ROM), flash memory, etc.), or some combination of the two. This most basic configuration is illustrated in FIG. 12 by dashed line 1206.

Computing device 1200 may have additional features/functionality. For example, computing device 1200 may include additional storage (removable and/or non-removable) including, but not limited to, magnetic or optical disks or tape. Such additional storage is illustrated in FIG. 12 by removable storage 1208 and non-removable storage 1210.

Computing device 1200 typically includes a variety of computer readable media. Computer readable media can be any available media that can be accessed by the device 1300 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 1204, removable storage 1208, and non-removable storage 1210 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1200. Any such computer storage media may be part of computing device 1200.

Computing device 1200 may contain communication connection(s) 1212 that allow the device to communicate with other devices. Computing device 1200 may also have input device(s) 1214 such as a keyboard, mouse, pen, voice input device, touch input device, etc. Output device(s) 1216 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method for providing a vaccination record comprising:
determining that a patient received a vaccination by a computing device;
in response to the determination, generating vaccination data for the patient by the computing device, wherein the vaccination data indicates the received vaccine;
receiving a request for a vaccination record from the patient by the computing device;
in response to the request, retrieving the vaccination data associated with the patient by the computing device;
generating the vaccination record using the vaccination data by the computing device, wherein the vaccination record comprises a code; and
presenting the generated vaccination record by the computing device.

2. The method of claim 1, wherein presenting the generated vaccination record comprises presenting the generated vaccination record to the patient or a third party verifier.

3. The method of claim 1, wherein determining that the patient received the vaccination comprises receiving an insurance claim associated with the vaccination and determining that the patient received the vaccination based on the insurance claim.

4. The method of claim 1, wherein determining that the patient received the vaccination comprises receiving an indication of payment associated with the vaccination and determining that the patient received the vaccination based on the indication of payment.

5. The method of claim 1, wherein determining that the patient received the vaccination comprises receiving an administrative trigger associated with the vaccination and determining that the patient received the vaccination based on the administrative trigger.

6. The method of claim 1, wherein the vaccination data comprises an identifier of a location of where the vaccination was administered, an identifier of a healthcare provider that administered the vaccine, a type of the vaccine, and a dose number of the vaccine.

7. The method of claim 1, wherein the code is a QR code.

8. The method of claim 1, wherein the request for the vaccination includes a telephone number associated with the patient.

9. The method of claim 1, wherein the code comprises a link to the vaccination record.

10. The method of claim 1, wherein the vaccination record is displayed in a webpage to the patient on a computing device associated with the patient.

11. The method of claim 1, wherein the vaccination record is displayed in an app running on a computing device associated with the patient.

12. The method of claim 1, further comprising:
retrieving a code from the vaccination record associated with the patient by a computing device;
using the code, retrieving a challenge question for the patient by the computing device;
providing the challenge question to the patient by the computing device;
receiving an answer to the challenge question from the patient by the computing device;
verifying that the answer is correct by the computing device; and
in response to the verification, determining that the patient has received the vaccine by the computing device.

13. A method for verifying a patient associated with a vaccination record comprising:
receiving a code from a vaccination record associated with a patient by a computing device;
using the code, retrieving a challenge question for the patient by the computing device;
providing the challenge question to the patient by the computing device;
receiving an answer to the question from the patient by the computing device;
verifying that the answer is correct by the computing device; and
in response to the verification, determining that the patient has received a vaccine associated with the vaccination record by the computing device.

14. The method of claim 13, wherein, retrieving the challenge question for the patient comprises:
extracting a link from the code by the computing device;
following the link to a webpage by the computing device; and
retrieving a challenge question from the webpage by the computing device,

15. The method of claim 13, wherein the code is a QR code or a bar code.

16. A system for providing a vaccination record comprising:
at least one computing device; and
a computer-readable medium storing computer-executable instructions that when executed by the at least one computing device cause the at least one computing device to:
determine that a patient received a vaccination
in response to the determination, generate vaccination data for the patient, wherein the vaccination data indicates the received vaccine;
receive a request for a vaccination record from the patient;
in response to the request, retrieve the vaccination data associated with the patient;
generate the vaccination record using the vaccination data, wherein the vaccination record comprises a code; and
present the generated vaccination record.

17. The system of claim 16, wherein determining that the patient received the vaccination comprises receiving an insurance claim associated with the vaccination and determining that the patient received the vaccination based on the insurance claim.

18. The system of claim 16, wherein determining that the patient received the vaccination comprises receiving an indication of payment associated with the vaccination and determining that the patient received the vaccination based on the indication of payment.

19. The system of claim 16, wherein determining that the patient received the vaccination comprises receiving an administrative trigger associated with the vaccination and determining that the patient received the vaccination based on the administrative trigger.

20. The system of claim 15, wherein presenting the vaccination record comprises presenting the vaccination record to the patient or a third-party verifier.
